(19) United States
(12) Patent Application Publication
(10) Pub. No.: US 2006/0035976 A1
(43) Pub. Date: Feb. 16, 2006

(54) Methods of preventing headaches with norepinephrine precursors

(76) Inventor: Stephen J Peroutka, Hillsborough, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/523,843

(22) PCT Filed: Aug. 8, 2003
(86) PCT No.: PCT/US03/24691

§ 371 Date: Aug. 30, 2005

Related U.S. Application Data
(60) Provisional application No. 60/401,775, filed on Aug. 8, 2002.
(60) Provisional application No. 60/443,549, filed on Jan. 30, 2003.

(51) Int. Cl. A61K 31/198

(52) U.S. Cl. 514/567

(57) ABSTRACT

The present invention relates to methods of preventing headaches in a subject in need thereof, comprising, administering effective amounts of a norepinephrine precursor, threo-3-(3,4-dihydroxyphenyl)serine, a derivative thereof, or a pharmaccutically-acceptable salt thereof. The methods can be used to prevent migraine, tension-type, premenstrual, and other types of headaches.

This application claims the benefit of U.S. Provisional Application Ser. No. 60/401,775, filed Aug. 8, 2002, and U.S. Provisional Application Ser. No. 60/443,549, filed Jan. 30, 2003, which are hereby incorporated by reference in their entirety.

### DESCRIPTION OF THE INVENTION

Headache is a common medical complaint that involves the sensation of pain in and/or around the head and neck. Migraine is a specific subtype of headache that has been defined as a recurrent, often familial, symptom complex of periodic attacks of vascular headache. Migraines affect approximately 17% of adult women and 6% of adult men (Stewart et al., Neurology, 1994, 44 (suppl. 4), 517-523). Headache is the most commonly recognized symptom of migraine, but prominent changes also occur in gastrointestinal activity, cardiovascular reflexes, vascular smooth muscle function, cutaneous blood flow, platelet function and pupillary reactivity, indicating involvement with a number of different physiological pathways. Non-migrainous headaches, such as tension-type headaches, can be defined as recurrent episodes of headache lasting minutes to days. The pain is typically pressing or tightening in quality, of mild to moderate intensity, bilateral in location and does not worsen with routine physical activity. Nausea is absent, but photophobia or phonophobia may occur.

Although chronic and recurring headache syndromes are experienced by a large segment of the world population, there are no highly effective treatments for preventing or reducing the frequency and intensity of their reoccurrence. One of the difficulties in choosing a prevention strategy is that the etiology of headache is still poorly understood. In migraineurs, for instance, disturbances have been observed in different physiological systems, involving a range of seemingly unrelated symptoms, including, e.g., increased sensitivity to adrenergic and dopaminergic agonists, increased blood flow in the extremities, decreased cutaneous blood flow in the face, mild pupillary asymmetries, deficient cardiovascular reflexes, hypersensitivity to norepinephrine (NE), dopamine (DA) and 5-hydroxytryptamine (5-HT), increases in neuropeptide Y (NPY), and induction of platelet aggregation and degranulation. Accordingly, multiple classes of drugs have been used to treat migraine, such as serotonin agonists (e.g., sumatriptan) and antagonists, tricyclic antidepressants, calcium channel blockers, and beta-blockers. There has been no consensus to date on which physiological system is responsible for initiating headaches, and producing the subsequent pain and symptoms associated with it.

While not wishing to be bound by any theory, it is proposed herein that an acute migraine attack, and other headaches, results from the biochemical and physiological changes that occur within the nervous system following “excessive” sympathetic nervous system (SNS) activity. The primary biochemical change within the SNS involves a relative depletion of terminal neurotransmitter stores of norepinephrine (NE) in the SNS. The headache phase of the migraine begins when the effects of SNS co-transmitters such as DA, prostaglandins (PGs) and adenosine triphosphate (ATP) predominate over NE sympathetic neurotransmission. This physiological state exacerbates the relative deficiency of NE via negative feedback loops on NE terminals. The resulting increase in DA, PGs and adenosine leads to a local activation of visceral afferent pain fibers. Clinical signs of a NE-depleted SNS during the later stages of an attack include low levels of plasma NE, nasal stuffiness and diuresis. Migraineurs and other individuals prone to headache can exhibit peripheral SNS dysfuction, e.g., resulting in reduced SNS activity, that under stressful and other conditions produces a headache.

Accordingly, the present invention relates to a methods of preventing headaches in a subject in need thereof, comprising, administering to the subject an amount of a norepinephrine precursor which is effective to prevent the headaches. By the term “preventing,” it is meant that the norepinephrine precursor or other active agent (see below for other active agents) reduces the frequency, severity, intensity, and/or duration of a headache when administered prior to the headache's onset. An amount of the active agent that is effective in preventing the headache is any amount that reduces the frequency, severity, intensity, and/or duration of headaches in a subject being treated. The specific dose level and frequency of dosage may vary, and can depend upon a variety of factors, including the activity of the specific active agents, their metabolic stability and length of action, half-life, rate of excretion, mode and time of administration, and the age, body weight, general health, gender, diet, and severity, intensity, and frequency of the onset of the headache, of the particular condition of the subject undergoing therapy. An active agent in accordance with the present invention can be immediately effective in achieving prophylaxis, or can reach its maximal effect after multiple, regular doses, e.g., one or more doses a day for a week, two weeks, a month, etc.

Although headaches can be prevented in any subject, certain subjects can be excluded from the present invention, e.g., subjects who have Parkinson's disease, subjects who have brain degenerative disorders, subjects who have previously been medicated and/or are currently being medicated with Parkinson's drugs, such as L-DOPA, benserazide, and/or amantadine, subjects who experience cluster headaches, etc. Thus, the present invention relates to methods of preventing headaches in a subject in need thereof, comprising, administering to the subject an amount of a norepinephrine precursor which is effective to prevent the headaches, with the proviso, e.g., that said subject does not have Parkinson's disease.

Any norepinephrine precursor that is effective in preventing headaches can be used. A norepinephrine precursor comprises any compound which is converted into the norepinephine neurotransmitter. These include, e.g., a substrate of the enzyme dopa decarboxylase that can be converted to norepinephrine, such as threo-3-(3,4-dihydroxyphenyl)serine, or a substrate of the enzyme dopamine beta-hydroxylase that can be converted to norepinephrine, such as dopamine. Active agents of the present invention can be selected such that they are selective for the norepinephrine system, i.e., that they do not have significant effects on other neurotransmitter pathways.

Headaches that can be prevented in accordance with the present invention include, e.g., migraine without aura, migraine with aura, tension-type headache, premenstrual-headache, etc. Migraine without aura can be associated with, e.g., hemicranial or bilateral pain, pulsating head pain, steady nonpulsatile head pain, nausea, vomiting, photophobia, phonophobia, and osmophobia. Migraine with aura has similar symptoms, but subjects also experience aura. Subjects with tension-type headaches can experience, e.g., bilateral, occipital, or frontal head pain, aching, tight, and squeezing head pain, and nausea. In addition to reducing the frequency of headaches, methods of the present invention can reduce, eliminate, decrease, etc., the severity of one or more the aforementioned symptoms.

The present invention also relates to methods of preventing headaches in a subject in need thereof, comprising, administering to said subject an amount of threo-3-(3,4-dihydroxyphenyl) serine, a derivative thereof, or a pharmaceutically-acceptable salt thereof, which is effective to prevent said headaches. As for any of the methods of the present invention, subject can be excluded, e.g., with the proviso that said subject does not have Parkinson's disease.

Threo-3-(3,4-dihydroxyphenyl)serine (also known as threo-DOPS or DOPS or droxidopa) is a synthetic amino acid precursor of NE (Freeman R., Clin. Neuropharm., 14, 296-304, 1991). DOPS is directly converted to NE via the actions of dopa decarboxylase (DDC) (also known as L-aromatic amino acid decarboxylase or AAAD). It has four stereoisomers, L-threo-DOPS, D-threo-DOPS, L-erythro-DOPS, and D-erythro-DOPS. Of the four, L-threo-DOPS is preferred, but a racemate can also be used. Peak plasma levels of DOPS occurs 3 hour after oral ingestion whereas pealk NE levels occur 5 hours after ingestion. Increased plasma levels of both molecules remain at least 12 hours after oral administration of DOPS (S Suzuki T, Higa S, Sakoda S, Ueji M, Hayashi A, Takaba Y, Nakajima A.; Eur J Clin Pharmacol 1982;23(5):463-8). Specific uptake of DOPS has also been demonstrated in microvessel preparations (Hardebo J E, Falck B, Owman C.Acta Physiol Scand 1979 October;107(2):161-7). Although threo-3-(3,4-dihydroxyphenyl)serine is known as a norepinephrine precursor, the present invention includes any preventative or prophylactic effect against headaches, regardless of its mechanism of action or how it is achieved.

DOPS has been used to treat motor or speech disturbances (e.g., U.S. Pat. No. 5,656,669), Parkinson's disease, cerebral ischemia (e.g., EP 887 078), urinary incontinence (e.g., U.S. Pat. No. 5,266,596, orthostatic hypotension (Freeman, 1991), and pain (e.g., U.S. Pat. No. 5,616,618; EP 681 838), but not for preventing headaches. Takagi (e.g., Eur. Neuropsychopharm., 6: 43-47, 1996) discloses alleged analgesic effects of L-DOPS (100 mg), including in a patient experiencing a cluster headache, but does not describe preventing a headache occurrence. Indo, Shinkei Naika. Neurol. Med., 21:232-233, 1984, describes administering Leehreo-DOPS (100 mg/day) to a 43 year-old female with Parkinson's disease who was concurrently being treated with L-DOPA, benserazide, and amantadine. The patient allegedly reported that the migraine attacks were resolved. The patient classes disclosed in both Takagi and Indo can be excluded as subjects from the present invention. Any effective amount of threo-3-(3,4-dihydroxyphenyl)serine can used, e.g., from about 10 mg to about 600 mg per day, about 50 mg to about 150 mg per day, etc. Effective amounts can be determined routinely, and may vary depending upon the age, health, gender, and weight of a patient, as well as the severity, frequency, and duration of the headaches. Amounts can be administered in a multiple doses over the course of the day, e.g., in order to achieve a prophylactic effect. It can also be administered with a decarboxylase inhibitor, e.g., benserazide or carbidopa.

Threo-3-(3,4-dihydroxyphenyl) serine can be prepared according to any suitable method. These processes include those described in, e.g., U.S. Pat. Nos. 4,480,109, 4,562,263 and 5,864,041. It can be used as a racemate or optically active isomer, e.g., L-threo-DOPS.

Pharmaceutically-acceptable salts of threo-3-(3,4-dihydroxyphenyl)serine can also be used, including addition salts, e.g., inorganic acids, such as hydrochloric acid, hydrobromic acid, and sulfuric acid, and organic acids, such as famaric acid, citric acid, tartaric acid, and succinic acid.

Any pharmacologically active derivative of threo-3-(3,4-dihydroxyphenyl)serine can be used. These include, e.g., N-methyl-3-(3,4-dihydroxyphenyl)serine alkyl esters, such as N-methyl-D,L-threo-3-(3,4-dihydroxyphenyl)serine and N-methyl-L-threo-3-(3,4dihydroxyphenyl)serine, lower allcyl esters, methyl esters, ethyl esters, n-propyl esters, isopropyl esters, etc., as described in U.S. Pat. No. 5,288,898.

An active agent of the present invention can be administered alone, or in combination with other agents. In certain embodiments of the invention, an agent such as a norepinephrine precursor, threo-3-(3,4-dihydroxyphenyl)serine, or derivatives thereof, is administered without another pharmacologically active agent, but comprising, e.g., pharmaceutically acceptable carriers, i.e., consisting essentially of administering said norepinephrine precursor, threo-3-(3,4-dihydroxyphenyl)serine, or derivatives thereof. Methods also include administering an active agent with other pharmacologically active agents, as long as a serotonin precursor is not administered (e.g., with the proviso that a serotonin precursor as disclosed in U.S. Pat. No. 5,939,076 are not administered). Methods also include administering an active agent with other pharmacologically active agents, as long as L-DOPA, benserazide, and/or amantadine are not administered (e.g., with the proviso that benserazide is not administered). See, Indo, 1984. Other peripheral decarboxylase inhibitors can also be excluded, such carbidopa, as well as other agents that are used to increase the central availability of L-DOPA.

It is well known that identifiable triggering factors and events can result in a headache in a susceptible subject. Specific examples that trigger migraines include environmental changes, such as stress, sleep patterns, hormonal shifts, premenstrual tension, and hypoglycemia. Likewise, tension-type headaches can be initiated by stress, exhaustion, alcohol, depression, anxiety, etc. Active agents of the present invention can prevent a headache in such an individual, despite the presentation of the typical triggering factors. Along these lines, the present invention relates to methods of preventing headaches in a subject in need thereof, comprising, administering to said subject an amount of a norepinephrine precursor, threo-3-(3,4-dihydroxyphenyl)serine, a derivative thereof, or a pharmaceutically-acceptable salt thereof, which is effective to increase the threshold for triggering-said headaches, thereby preventing said headaches. A susceptible subject may experience certain factors and events (e.g., changes in the subject's environment and physical condition) that trigger or initiate the headache attack. The active agents disclosed herein increase the subject's resistance to the factors and/or events, making it less likely that they will precipitate a headache. As a result, the frequency of the attacks are reduced. Thus, the subject's threshold for triggering a headache is increased and headaches can be prevented.

Active agents in accordance with the present invention can be administered in any form by any effective route, including, e.g., oral, parenteral, enteral, intraperitoneal, topical, transdermal (e.g., using any standard patch), ophthalmic, nasally, local, non-oral, such as aerosal, spray, inhalation, subcutaneous, intravenous, intramuscular, buccal, sublingual, rectal, vaginal, intra-arterial, and intrathecal, etc. It can be administered alone, or in combination with any ingredient(s), active or inactive.

Active agents can be combined with any pharmaceutically acceptable carrier. By the phrase, “pharmaceutically acceptable carriers,” it is meant any pharmaceutical carrier, such as the standard carriers described, e.g., Remington's Pharmaceutical Science, Eighteenth Edition, Mack Publishing company, 1990. Examples of suitable carriers are well known in the art and can include, but are not limited to, any of the standard pharmaceutical carriers such as a phosphate buffered saline solutions, phosphate buffered saline containing Polysorb 80, water, emulsions such as oil/water emulsion and various type of wetting agents. Other carriers may also include sterile solutions, tablets, coated tablets pharmaceutical and capsules. Typically such carriers contain excipients such as such as starch, milk, sugar, certain types of clay, gelatin, stearic acid or salts thereof, magnesium or calcium stearate, talc, vegetable fats or oils, gums, glycols. Such carriers can also include flavor and color additives or other ingredients. Compositions comprising such carriers are formulated by well known conventional methods.

Suitable pharmaceutically acceptable carriers include but are not limited to water, salt solutions, alcohols, gum arabic, vegetable oils, benzyl alcohols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose and the like. Other additives include, e.g., antioxidants and preservatives, coloring, flavoring and diluting agents, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxppropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as microcrystalline cellulose, citric acid, dextrin, dextrose, liquid glucose, lactic acid, lactose, magnesium chloride, potassium metaphosphate, starch, and the like.

The active agent or the novel composition of this invention may be in a form suitable for oral use, for example, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions and typically such compositions contain one or more agents selected from the group consisting of sweetening agents, flavoring agents, coloring agents and preservatives in order to provide pharmaceutically elegant and palatable preparations. These excipients may be for example, diluents such as lactose, calcium carbonate, sodium carbonate, calcium phosphate or sodium phosphate; granulating and disintegrating agents, for example, corn starch or alginic acid; binding agents, for example starch, gelatin or acacia, and lubricating agents, for example, magnesium stearate, stearic acid or talc.

The tablets may be uncoated or they may be coated. Coating can be included to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or miscible solvents such as propylene glycol, PEGs and ethanol, or an oil medium, for example peanut oil, liquid paraffin or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, tragacanth and acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The individual agents or the pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxy-ethylene sorbitan monooleate. The emulsions may also contain sweetening and flavouring agents.

The pharmaceutical compositions may be in the form of a sterile injectable aqueous or oleagenous suspension. This suspension may be formulated according to the known art using those suitable dispersing or wetting agents and suspending agents which have been mentioned above.

Injectable compositions are typically in the form of sterile solutions or suspensions, which include the active ingredient in a parenterally-acceptable diluent. Among these are sterile water, dextrose 5% in water (D5W), Ringer's solution and isotonic saline, as well as mixtures thereof. Cosolvents such as ethanol, propylene glycol or polyethylene glycols may also be used. Sterile, injectable oil is occasionally employed as a solvent or suspending medium in intramuscular preparations. A representative example is peanut oil. In addition, fatty acids such as oleic acid, preservatives, buffers and local anesthetics find use in the preparation of intramuscular injectables.

The active ingredient may also be administered rectally or intravaginally as suppositories. These can be prepared by mixing the drug with a suitable non-irritating excipient which is solid at ordinary room temperature but molten at normal or elevated body temperature. Examples of such materials include cocoa butter and polyethylene glycols.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. The entire disclosure of all applications, patents and publications, cited above are hereby incorporated by reference in their entirety.

1. A method of preventing headaches in a subject in need thereof, comprising,
administering to said subject an amount of threo-3-(3,4-dihydroxyphenyl)serine, or a pharmaceutically-acceptable salt thereof, which is effective to prevent said headaches, with the proviso that said subject does not have Parkinson's disease.

2. A method of claim 1, consisting essentially of administering threo-3-(3,4-dihydroxyphenyl)serine, or a pharmaceutically-acceptable salt thereof.

3. A method of claim 1, with the proviso that a serotonin precursor is not administered.

4. A method of claim 1, with the proviso that benserazide is not administered.

5. A method of claim 1, wherein said threo-3-(3,4-dihydroxyphenyl) serine is the L-form.

6. A method of claim 1, wherein said headaches are migraine headaches.

7. A method of claim 1, wherein said headaches are tension-type headaches or premenstrual headaches.

8. (canceled)

9. A method of claim 1, wherein said preventing is reducing the frequency of said headaches.

10. A method of preventing headaches in a subject in need thereof, comprising,
administering to said subject an amount of a threo-3-(3,4-dihydroxyphenyl) serine derivative, or a pharmaceutically-acceptable salt thereof, which is effective to prevent said headaches, with the proviso that said subject does not have Parkinson's disease.

11. A method of claim 10, consisting essentially of administering a threo-3-(3,4-dihydroxyphenyl)serine derivative, or a pharmaceutically-acceptable salt thereof.

12. A method of claim 10, with the proviso that a serotonin precursor is not administered.

13. A method of claim 10, wherein said threo-3-(3,4-dihydroxyphenyl)serine derivative is the L-form.

14. A method of claim 10, wherein said headaches are migraine headaches.

15. (canceled)

16. A method of claim 10, wherein said preventing is reducing the frequency of said headaches.

17. A method of claim 10, wherein said derivative is an N-methyl-3-(3,4-dihydroxyphenyl)serine alkyl ester.

18. A method of preventing headaches in a subject in need thereof, comprising,
administering to said subject an amount of a norepinephrine precursor which is effective to prevent said headaches, with the proviso that a serotonin precursor is not administered and that said subject does not have Parkinson's disease.

19. A method of claim 18, consisting essentially of administering said norepinephrine precursor.

20. A method according to claim 18, wherein said norepinephrine precursor is threo-3-(3,4-dihydroxyphenyl)serine.

21. A method according to claim 18, wherein said norepinephrine precursor is the L-form of threo-3-(3,4-dihydroxyphenyl) serine.

22. A method according to claim 18, wherein said norepinephrine precursor is a substrate of the enzyme dopa decarboxylase that can be converted to norepinephrine.

23. A method according to claim 18, wherein the norepinephrine precursor is a substrate of the enzyme dopamine beta-hydroxylase that can be converted to norepinephrine.

24. A method of claim 18, wherein said headaches are tension type migraine headaches.

25. (canceled)

26. A method of claim 18, wherein said preventing is reducing the frequency of said headaches.

27. A method of preventing headaches in a subject in need thereof, claim 1, wherein said amount is effective to increase the threshold for triggering said headaches, thereby preventing said headaches.

28. A method of claim 27, wherein said threo-3-(3,4-dihydroxyphenyl) serine is the L-form.

29. A method of claim 27, wherein said headaches are migraine headaches.

30. (canceled)

31. (canceled)

32. A method of claim 26, wherein said preventing is reducing the frequency of said headaches.

33. A method of claim 1, wherein threo-3-(3,4-dihydroxyphenyl) serine, or a pharmaceutically acceptable addition salt thereof, is administered with a decarboxylase inhibitor.
